(19) United States
(12) Patent Application Publication
(10) Pub. No.: US 2025/0000766 A1
(43) Pub. Date: Jan. 2, 2025

(54) ANTIMICROBIAL ESTERS FOR SKIN AND SCALP CARE

(71) Applicant: SYMRISE AG, Holzminden (DE)

(72) Inventors: Florian GENRICH, Dassel (DE); Sabine LANGE, Holzminden (DE); Steffen NORDZIEKE, Göttingen (DE); Jessica GRIEGER, Höxter (DE)

(73) Assignee: SYMRISE AG, Holzminden (DE)

(21) Appl. No.: 18/687,048

(22) PCT Filed: Aug. 27, 2021
(86) PCT No.: PCT/EP2021/073770

§ 371 Date: Feb. 27, 2024

(51) Int. Cl. A61K 8/37; A61Q 5/00

(52) U.S. Cl. CPC A61K 8/375; A61Q 5/006

(57) ABSTRACT

The present invention relates to fatty acid esters for use in the treatment of an excess of Malassezia on the skin surface. Furthermore, the present invention relates to mixtures comprising one or more of such fatty acid esters for use in the treatment of an excess of Malassezia on the skin surface. Additionally, the present invention relates to compositions comprising one or more of such fatty acid esters or such mixtures for use in the treatment of an excess of Malassezia on the skin surface. Moreover, the present invention relates to cosmetic, non-therapeutic uses of such a fatty acid ester, such a mixture or such a composition.

### FIELD OF THE DISCLOSURE

The present invention relates to fatty acid esters for use in the treatment of an excess of Malassezia on the skin surface. Furthermore, the present invention relates to mixtures comprising one or more of such fatty acid esters for use in the treatment of an excess of Malassezia on the skin surface. Additionally, the present invention relates to compositions comprising one or more of such fatty acid esters or such mixtures for use in the treatment of an excess of Malassezia on the skin surface. Moreover, the present invention relates to cosmetic, non-therapeutic uses of such a fatty acid ester, such a mixture or such a composition.

Further aspects of the present invention will arise from the description below, in particular from the examples, as well as from the attached patent claims.

### BACKGROUND

Malassezia is a genus of fungi and is naturally found on the skin surfaces of many animals, including humans. It is involved in the pathogenesis of a variety of diseases, in particular skin diseases, and undesired conditions such as, for example, in pityriasis versicolor, seborrheic dermatitis and dandruff.

Pityriasis versicolor (or tinea versicolor) is a condition characterized by a skin eruption, typically on the trunk and proximal extremities of a subject. Pityriasis versicolor is known to be caused by Malassezia, such as M. globosa.

Seborrhoeic dermatitis (or seborrhoea) is a long-term skin disorder typically characterized by red, scaly, greasy, itchy, and inflamed skin, particularly the scalp. Seborrhoeic dermatitis is known to be caused by Malassezia, such as M. globosa, M. restricta, M. pachydermatis and M. sympodialis.

Dandruff is a skin condition, which is characterized by flaking and often mild itchiness of the skin, particularly the scalp. Dandruff is often referred to as the mild form of seborrheic dermatitis, however, without an inflammation. Thus, dandruff is considered as a cosmetic skin condition.

Short-chain and medium-chain fatty acids display good antimicrobial activity against Malassezia. However, their practical use in topical therapy is limited by their (i) intense smell, (ii) skin irritation, (iii) lack of skin-substantiveness and (iv) the difficulties they pose during formulation into products.

Various esters of said fatty acids do not display these disadvantageous properties. It is known that Malassezia is able to cleave such esters and to release the fatty acids, which leads to a “self-kill” of the fungi. However, the hydrolysis rates of esters through Malassezia enzymes vary strongly depending on the alcohol component of the esters.

In DE 42 37 367 A1 fatty acid esters are described as antimycotic agents. These esters are preferably selected from the group of hexyl laurate, isopropyl stearate, glyceryl monolaurate, caprylic acid triglyceride and capric acid triglyceride.

SU 1286204 A1 discloses the use of a mixture of mono-(50-60%), di-(30-35%) and triesters (10-15%) of glycerol and undecylenic acid to give antimicrobial properties to a cosmetic base.

In addition to already known antimycotic agents, there is a constant need of novel antimycotic agents, preferably with a broad range of application, to provide a possibly high flexibility when choosing particular antimycotic agents for various applications.

DE 10 2013 009 616 A1 describes that certain fatty acid esters have an antimycotic effect against Malassezia. However, in DE 10 2013 009 616 A1, lower alcohols (methyl to butyl alcohol) are presented as preferred alcohol parts of the esters.

Furthermore, Mayser et al., Hydrolysis of Fatty Acid Esters by Malassezia furfur: Different Utilization Depending on Alcohol Moiety, Acta Derm Venereol, 1995, 75:105-109 describes that lower alcohols are preferably advantageous alcohol parts of esters with an antimycotic effect, since these alcohols provide a particularly fast hydrolysis of the ester. Higher alcohols are mentioned as disadvantageous and are considered to be only used in case no hydrolysis of the ester is desired. However, as described above, to provide a release of the fatty acid, which leads to a “self-kill” of Malassezia, the hydrolysis of the alcohol part and the fatty acid part of the ester is necessary.

Thus, in the state of the art, lower alcohols are considered to be most useful alcohol parts of esters with antimycotic effect against Malassezia. However, higher alcohols are considered disadvantageous or even non-functional.

However, there is a constant need for novel agents with an antimycotic effect, particularly an antimycotic effect against Malassezia. Furthermore, to overcome the limitation to lower alcohols, as described above, it would be desirable to find a way to also utilize higher alcohols for providing esters with an antimycotic effect.

It was thus an object of the present invention to provide further effective and compatible active agents for the treatment of Malassezia-associated diseases and undesired conditions, preferably of dandruff, on human scalp.

### DESCRIPTION

The primary object of the present invention is solved by a fatty acid ester, wherein the fatty acid ester is an ester of caprylic acid and a polyol, wherein the ester carries at least 3 hydroxyl groups at the polyol residue, for use in the treatment of an excess of Malassezia on the skin surface, preferably on the scalp, of mammals, preferably of humans.

It was surprisingly found that esters of caprylic acid and a polyol provided a particularly good effect against Malassezia, when the ester carries at least 3 hydroxyl groups at the polyol residue.

Preferably, the ester for use according to the invention carries 3 to 6 hydroxyl groups at the polyol residue, preferably 3 to 5 hydroxyl groups at the polyol residue, particularly preferably 3 or 4 hydroxyl groups at the polyol residue.

It is preferred that the number of hydroxyl groups at the polyol residue is determined at a pH of 7 and under standard conditions (i.e. 20° C., 1.013 bar), wherein the respective ester is provided, preferably dissolved, in water.

The esters for use according to the invention are esters of caprylic acid and a polyol. Caprylic acid and the polyol form an ester bond, particularly the OH-part of the carboxylic acid group of caprylic acid and a hydroxyl group of the polyol form an ester bond via esterification, and provide an ester as defined herein. Preferably, an ester for use according to the invention may have more than one ester bond. Thus, it is preferred that the ester for use according to the invention is a monoester or a diester or a mixture of a monoester and a diester.

Thus, the term “polyol residue” of an ester, as used herein, is meant to be understood as the part of the ester, which originates from the polyol.

Likewise, the term “fatty acid residue” of an ester, as used herein is meant to be understood as the part of the ester, which originates from the fatty acid. At least for one ester bond, this fatty acid is caprylic acid, particularly preferably for each ester bond the fatty acid is caprylic acid.

Against the concept imparted by the state of the art, it was surprisingly found that higher alcohols may be utilized to provide an ester with antimycotic effect, at least when the resulting ester carries at least 3 hydroxyl groups at its polyol residue.

Thus, it is preferred that the polyol residue of the ester carries at least 5 carbon atoms, preferably 5 to 10 carbon atoms, particularly preferably 5 to 9 carbon atoms.

Preferably, the polyol residue may carry one or more hydroxyl groups, which additionally form one or more ester bond(s) via esterification with another fatty acid, preferably wherein the fatty acid residue of the, one or more or all additional ester bonds originates from caprylic acid. Thus, it is preferred that the ester has more than one ester bond.

Consequently, it is preferred that the ester is a monoester or a diester or a mixture of a monoester and a diester, preferably wherein in case the ester is a diester or a mixture of a monoester and a diester, both fatty acid residues of the diester originate from caprylic acid.

Caprylate, as used herein, refers to an ester of caprylic acid (CAS Registry Number of caprylic acid: 124-07-2; also known as octanoic acid).

The term “the ester is a mixture of a monoester and a diester” is meant to be understood such that the ester is a mixture of esters, comprising or consisting of one or more monoester(s) and one or more diester(s), preferably wherein the monoesters (if more than one is present) have the same polyol residue, but the ester bond is at a different hydroxyl group of the polyol residue, and/or preferably wherein the diesters (if more than one is present) have the same polyol residue, but at least one ester bond is at a different hydroxyl group of the polyol residue, and/or preferably wherein the monoester(s) and the diester(s) have the same polyol residue, but the diester(s) have one more ester bond at the polyol residue, preferably wherein none or one of the ester bonds of the diester(s) is at the same position as in the monoester(s).

Examples for such mixtures of a monoester and a diester are xylityl sesquicaprylate or sorbitan sesquicaprylate, which are known to be present as mixtures of a corresponding monoester and a corresponding diester, as described above.

Furthermore, it is preferred that the ester is selected from the group consisting of xylityl caprylate, preferably xylityl sesquicaprylate, polyglyceryl-3 caprylate and sorbitan caprylate, preferably sorbitan sesquicaprylate,

preferably wherein the ester is selected from the group consisting of xylityl sesquicaprylate, polyglyceryl-3 caprylate and sorbitan caprylate.

The compound “xylityl caprylate” is an ester of xylitol and one, two or more, preferably one caprylic acid(s), which is preferably described by INCI Monograph ID: 32988 and is preferably represented by the following chemical formula:

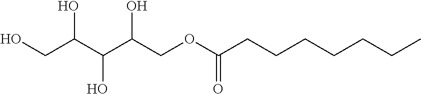 [SMILES: CCCCCCCC(=O)OCC(O)C(O)C(O)CO]

Preferably, the compound “xylityl caprylate” additionally or alternatively describes corresponding esters of xylitol and one, two or more, preferably one, caprylic acid(s) and salts thereof.

The compound “xylityl sesquicaprylate” is understood as a subgroup of xylityl caprylate, as described above, and is an ester of xylitol and one, two or more, preferably one, caprylic acid(s). Furthermore, xylityl sesquicaprilate is a mixture of one or more monoester(s) and one or more diester(s), as described above. Xylityl sesquicaprylate is preferably described by CAS-No. 181632-90-6 and is preferably represented by the following chemical formulae:

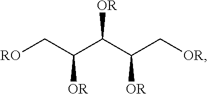

wherein one of R represents an octanoyl residue and the remaining R are hydrogen, together with

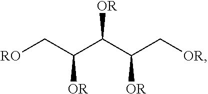

wherein two of R represent an octanoyl residue and the remaining R are hydrogen.

Preferably, the compound “xylityl sesquicaprylate” additionally or alternatively describes corresponding esters of xylitol and one, two or more, preferably one or two, caprylic acid(s) and salts thereof.

The compound “polyglyceryl-3 caprylate” is an ester of triglycerol and one, two or more, preferably one, caprylic acid(s), which is preferably described by CAS-No. 108777-93-1 and is preferably represented by the following chemical formula:

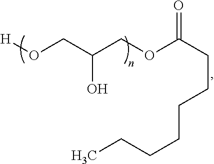

wherein n is 3.

Preferably, the compound “polyglyceryl-3 caprylate” additionally or alternatively describes corresponding esters of triglycerol and caprylic acid and salts thereof.

The compound “sorbitan caprylate” is an ester of hexitol anhydrides, anhydrosorbitol, dianhydrosorbitol, sorbitan or sorbitol and one, two or more, preferably one, caprylic acid(s), which is preferably described by CAS-No. 60177-36-8, additionally or alternatively by EC number 939-179-3, and is preferably represented by the following chemical formula:

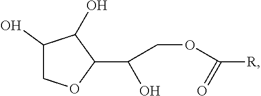

wherein R represents n-heptane.

Preferably, the compound “sorbitan caprylate” additionally or alternatively describes corresponding esters of sorbitan or sorbitol and one, two or more, preferably one, caprylic acid(s) and salts thereof.

The compound “sorbitan sesquicaprylate” is understood as a subgroup of sorbitan caprylate, as described above, and is an ester of hexitol anhydrides, anhydrosorbitol, dianhydrosorbitol, sorbitan or sorbitol and one, two or more, preferably one, caprylic acid(s). Furthermore, sorbitan sesquicaprylate is a mixture of one or more monoester(s) and one or more diester(s), as described above. Sorbitan sesquicaprylate is preferably described by CAS-No. 91844-53-0 and is preferably represented by the following chemical formulae:

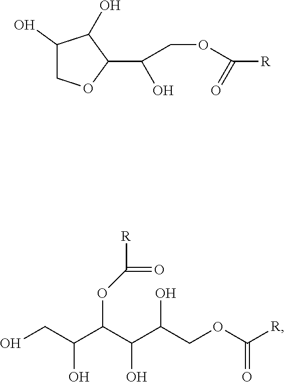

wherein R represents n-heptane.

Preferably, the compound “sorbitan sesquicaprylate” additionally or alternatively describes corresponding esters of sorbitan or sorbitol and one, two or more, preferably one, caprylic acid(s) and salts thereof.

The term “corresponding esters” as used for describing the above esters includes esters, in which the ester bond is formed at another hydroxyl group of the polyol residue, and/or preferably compounds, in which one or more further ester bond(s) are present at the polyol residue, as well as derivatives thereof. Furthermore, the term “corresponding esters” also includes any stereoisomer of the described compound(s).

Furthermore, the invention relates to a fatty acid ester selected from the group consisting of caprylyl caprylate (CAS Registry Number 2306-88-9 or INCI Monograph ID: 23727), coco-caprylate/caprate (CAS Registry Number 95912-86-0 (generic)), PEG-8 caprylate (INCI Monograph ID: 1970), dipropylene glycol caprylate (CAS Registry Number 220604. 16-0), propylene glycol caprylate (CAS Nr. 31565-12-5 and 68332-79-6), pyridoxine dicaprylate (CAS Registry Number 106483-4-9), glyceryl caprylate/caprate (INCI Monograph ID: 1080), methyl glucose caprylate/caprate (INCI Monograph ID: 15374), polyglyceryl-3 caprate/caprylate/succinate (CAS Registry Number 1798313-25-3 (generic)), polyglyceryl-2 caprylate (INCI Monograph ID: 4659), polyglyceryl-3 caprylate (CAS Registry Number 108777-93-1), polyglyceryl-4 caprylate (INCI Monograph ID: 24482), polyglyceryl-6 caprylate (INCI Monograph ID: 12561), polyglyceryl-10 caprylate (CAS Registry Number 51033-41-1), polyglyceryl-4 caprylate/caprate (INCI Monograph ID: 26393), polyglyceryl-6 caprylate/caprate (INCI Monograph ID: 26091), polyglyceryl-10 caprylate/caprate (INCI Monograph ID: 25512), propanediol dicaprylate/caprate (CAS Registry Number 1072005-10-7), xylityl caprate/caprylate (CAS Registry Number 2127407-61-6 (generic)) and xylityl caprylate (INCI Monograph ID: 32988),

for use in the treatment of an excess of Malassezia on the skin surface, preferably on the scalp, of mammals, preferably of humans.

The term “Malassezia”, as used herein, refers to one or more species of the genus Malassezia, preferably to one, two, three, four, five or more species of the genus Malassezia (as defined further below).

Malassezia species are naturally found on the skin surfaces of many animals, including humans. As these fungi require fatty acids to grow, they are most common in areas with many sebaceous glands, i.e. on the scalp, face, and upper part of the body. However, when the fungus grows too rapidly, the natural renewal of cells is disturbed and, for example, dandruff appears on the scalp along with an itching sensation.

As used herein, an excess of Malassezia on the skin surface, preferably on the scalp, of mammals, preferably of humans, thus relates to a situation where the total amount of Malassezia cells present on said skin surface leads to symptoms of skin disease such as, for example, redness, itching, dryness, flaking, greasiness, hypopigmentation and/or hyperpigmentation of the skin. The treatment of an excess of Malassezia on the skin surface preferably relates to an action that leads to a situation where the total amount of Malassezia cells present on said skin surface does not result in said symptoms of skin disease or wherein one, more or preferably all said symptoms are at least reduced.

Thus, preferably a use according to the present invention (as described herein, medical or cosmetic) is selected from or comprises the treatment and/or the prevention of one, more or all symptoms from the group consisting of redness, itching, dryness, flaking, greasiness, hypopigmentation and hyperpigmentation of the skin.

Within the framework of the present text, the term “on the skin surface” also includes the areas of the hair infundibulum, the junctional zone and/or the sebaceous glands, if applicable, where an excess of Malassezia may be present.

Furthermore, the present invention relates to a mixture comprising one or more fatty acid esters as defined herein, for use in the treatment of an excess of Malassezia on the skin surface, preferably on the scalp, of mammals, preferably of humans.

Surprisingly, it was found that the esters as defined herein provided a particularly good antimycotic effect against diverse Malassezia strains, such as M. globosa, M. restricta, M. pachydermatis and M. sympodialis.

As described above, the prior art suggests that lower alcohols are considered to be most useful alcohol parts of esters with antimycotic effect against Malassezia. However, higher alcohols are considered disadvantageous or even non-functional.

It was thus particularly surprising that esters of caprylic acid and a higher alcohol, as described herein, provided an equal or even improved antimycotic effect against diverse Malassezia strains compared to esters of caprylic acid and a lower alcohol with a known antimycotic effect, in case the higher alcohol is used in form of a polyol, particularly carrying at least 5 carbon atoms, and the resulting ester carries at least 3 hydroxyl groups at the polyol residue. This finding was particularly surprising and provides the advantage that also higher alcohols can be utilized for obtaining an ester with antimycotic effect against Malassezia, in case the respective alcohol, i.e. the alcohol with the corresponding chain length or, respectively number of carbon atoms, is used in form of a polyol as described above.

Particularly, an advantageous effect was observed with regard to M. globosa, M. restricta, M. pachydermatis and M. sympodialis.

Thus, the invention relates to a fatty acid ester as defined herein for use according to the invention or a mixture as defined herein for use according to the invention, wherein Malassezia is/are one or more species selected from the group consisting of M. globosa, M. restricta, M. pachydermatis and M. sympodialis.

Furthermore, the invention relates to a composition comprising a fatty acid ester as defined herein or a mixture as defined herein, wherein the composition is a leave-on or rinse-off personal care product, preferably selected from the group consisting of shampoos, preferably anti-dandruff shampoos, cleansing products, preferably shower gels, body washes and soaps, wet wipes, emulsions, preferably oil-in-water or water-in-oil emulsions, surfactant-based systems, tonics, preferably aqueous or aqueous/ethanolic/glycolic solutions and gels, leave-on and rinse-off conditioners, hair butters and waxes, beard care and conditioners,

for use in the treatment of an excess of Malassezia on the skin surface, preferably on the scalp, of mammals, preferably of humans, preferably wherein Malassezia is/are one or more species selected from the group consisting of M. globosa, M. restricta, M. pachydermatis and M. sympodialis.

It is advantageous to formulate the compositions as defined herein in a way that enables users to incorporate the treatment of an excess of Malassezia on their skin surface into their daily hair or skin care routine. This is achieved by, for example, formulating said compositions as hair or skin care products. By creating hair or skin care products comprising one or more fatty acid esters for use as defined herein, e.g. an anti-dandruff shampoo, the user can save time, money and effort by combining the treatment against an excess of Malassezia on the skin surface with their standard hair or skin care, e.g. washing hair. The rinse-off skin care product as defined herein may also, for example, be a shower gel and the leave-on skin care product may, for example, be a body cream or body lotion.

Preferably, the composition as defined herein further comprises
(i) one or more 1,2-alkane diol(s), preferably one or more 1,2-alkane diol(s) selected from the group consisting of 1,2-propane diol, 1,2-pentane diol, 1,2-hexane diol, 1,2-heptane diol, 1,2-octane diol, 1,2-nonane diol, 1,2-decane diol, 1,2-undecane diol, 1,2-dodecane diol and 1,2-tridecane diol, particularly preferably selected from the group consisting of 1,2-heptane diol and 1,2-nonane diol, and/or
(ii) one or more 2,3-alkane diol(s), preferably one or more 2,3-alkane diol(s) selected from the group consisting of 2,3-pentane diol, 2,3-hexane diol, 2,3-heptane diol, 2,3-octane diol, 2,3-nonane diol, 2,3-decane diol, 2,3-undecane diol, 2,3-dodecane diol, 2,3-tridecane diol, particularly preferably selected from the group consisting of 2,3-heptane diol and 2,3-nonane diol, and/or
(iii) one or more 1,3-alkane diol(s), preferably one or both 1,3-alkane diol(s) selected from the group consisting of 1,3-propane diol, 1,3-butane diol and 2-methyl-2,4-pentan diol, particularly preferably 1,3-propane diol,
   particularly preferably wherein the composition further comprises one or more 1,2-alkane diol(s), as described above, and one or more 2,3-alkane diol(s), as described above, and optionally one or more 1,3-alkane diol(s), as described above.
   particularly preferably wherein the composition further comprises one or more 1,2-alkane diol(s), as described above, and one or more 2,3-alkane diol(s), as described above, and optionally 1,3-propane diol.

It is further preferred that the composition further comprises
(i) one or more 1,2-alkane diol(s), preferably one or more 1,2-alkane diol(s) selected from the group consisting of 1,2-propane diol, 1,2-pentane diol, 1,2-hexane diol, 1,2-heptane diol, 1,2-octane diol, 1,2-nonane diol, 1,2-decane diol, 1,2-undecane diol, 1,2-dodecane diol and 1,2-tridecane diol, particularly preferably selected from the group consisting of 1,2-heptane diol and 1,2-nonane diol, and
(ii) one or more 2,3-alkane diol(s), preferably one or more 2,3-alkane diol(s) selected from the group consisting of 2,3-pentane diol, 2,3-hexane diol, 2,3-heptane diol, 2,3-octane diol, 2,3-nonane diol, 2,3-decane diol, 2,3-undecane diol, 2,3-dodecane diol, 2,3-tridecane diol, particularly preferably selected from the group consisting of 2,3-heptane diol and 2,3-nonane diol, and
(iii) optionally one or more 1,3-alkane diol(s), preferably one or both 1,3-alkane diol(s) selected from the group consisting of 1,3-propane diol, 1,3-butane diol and 2-methyl-2,4-pentan diol, particularly preferably 1,3-propane diol.

Particularly preferably, the composition further comprises one, two, three or all alkane diols selected from 1,2-heptanediol, 1,2-nonanediol, 2,3-heptanediol and 2,3-nonanediol and optionally one or more 1,3-alkane diol(s) as described above.

It was found that addition of one or more alkane diol(s), as described above, leads to a strong or even synergistic increase in antimicrobial activity of the esters, as defined above, against Malassezia.

Preferably, the weight ratio between the total weight of fatty acid ester(s) as defined herein and the total weight of 1,2-alkane diol(s) (as defined herein), 2,3-alkane diol(s) (as defined herein) and 1,3-alkane diol(s) (as defined herein), each as far as present, comprised in the composition as defined herein is from 20:1 to 1:20, more preferably from 10:1 to 1:10 further preferably from 5:1 to 1:5, particularly preferably from 2:1 to 1:2.

Moreover, it is preferred that the composition further comprises one or more additional active agent(s), preferably one or more antimicrobial agent(s), more preferably one or more active agent(s) selected from the group consisting of clotrimazole (CAS Registry Number 23593-75-1), bifonazole (CAS Registry Number 60628-96-8), miconazole (CAS Registry Number 22916-47-8), ketoconazole (CAS Registry Number 65277-42-1), fluconazole (CAS Registry Number 86386-73-4), climbazole (CAS Registry Number 38083-17-9), itraconazole (CAS Registry Number 84625-61-6), terbinafine (CAS Registry Number 91161-71-6), nystatin (CAS Registry Number 1400-61-9), amorolfine (CAS Registry Number 78613-35-1), ciclopirox (CAS Registry Number 29342-05-0), octopirox (CAS Registry Number 68890-66-4) and undecylenic acid (CAS Registry Number 112-38-9).

Preferably, the composition comprises fatty esters as defined herein in a range of from 0.001 to 25 wt.-%, preferably in a range of from 0.005 to 20 wt.-%, particularly preferably in a range of from 0.01 to 10 wt.-%, more preferably in a range of from 0.02 to 5 wt.-%, further preferably in a range of from 0.05 to 3 wt.-%, especially preferably in a range of from 0.1 to 2 wt.-%, even further preferably in a range of from 0.2 to 1.5 wt.-%, particularly preferably in a range of from 0.5 to 1.0 wt.-%, related to the total weight of the composition.

What is said herein with regard to fatty acid esters or mixtures, as defined herein, applies accordingly to compositions as defined herein, as far as applicable, and vice versa.

The invention further relates to a fatty acid ester as defined herein or a mixture as defined herein or a composition as defined herein,

for use in the treatment or reduction of dandruff on human skin, preferably on the human scalp, wherein the dandruff is caused by Malassezia, particularly preferably wherein Malassezia is/are one or more species selected from the group consisting of M. globosa, M. restricta, M. pachydermatis and M. sympodialis.

In case a subject suffers from dandruff, however the dandruff is the symptom of a medical condition such as pityriasis versicolor or seborrhoeic dermatitis or further inflammatory conditions in this regard, the treatment or reduction of dandruff is considered medical, since the symptom of a medical condition is treated or reduced.

Thus, it is preferred that the dandruff on human skin, preferably on the human scalp, is caused by or a symptom of pityriasis versicolor or seborrhoeic dermatitis or an inflammatory disorder causing dandruff. Preferably in this case, pityriasis versicolor or seborrhoeic dermatitis or the inflammatory disorder causing dandruff is caused by Malassezia, particularly preferably wherein Malassezia is/are one or more species selected from the group consisting of M. globosa, M. restricta, M. pachydermatis and M. sympodialis.

In addition to the medical conditions caused by Malassezia, these fungi also cause cosmetic conditions of the skin, particularly the scalp, such as dandruff.

Therefore, the esters, mixtures and compositions as defined herein may also be used in reducing the amount of Malassezia for cosmetic reasons.

Thus, the invention also relates to the cosmetic, non-therapeutic use of a fatty acid ester as defined herein or a mixture as defined herein or a composition as defined herein to reduce the amount of Malassezia on the skin surface, preferably on the scalp, of mammals, preferably of humans. Preferably, Malassezia is/are one or more species selected from the group consisting of M. globosa, M. restricta, M. pachydermatis and M. sympodialis.

The nature of the respective treatment, being medical or being cosmetic, non-therapeutic, depends on the condition to be counteracted. As described above, pityriasis versicolor and seborrhoeic dermatitis are considered medical conditions. In case a subject suffering from one of these conditions is treated with regard to the condition, the treatment is considered as medical. Furthermore, these conditions may include dandruff as a symptom. Treatment or reduction of dandruff in this case is considered medical. However, in case a subject is suffering from dandruff, but not from e.g. pityriasis versicolor or seborrhoeic dermatitis or further inflammatory conditions in this regard, the condition is considered as cosmetic, as described above. In this case, the counteraction is considered as a cosmetic, non-therapeutic treatment.

Consequently, the invention also relates to the cosmetic, non-therapeutic use of a fatty acid ester as defined herein or a mixture as defined herein or a composition as defined herein to avoid dandruff and/or to reduce the amount of dandruff on human skin, preferably on human scalp, wherein the dandruff is caused by Malassezia. Preferably, Malassezia is/are one or more species selected from the group consisting of M. globosa, M. restricta, M. pachydermatis and M. sympodialis.

What is said herein with regard to medical uses, as described herein, applies accordingly to cosmetic uses, as described herein, as far as applicable, and vice versa.

The term “reducing the amount of dandruff” as used herein (for medical or cosmetic counteractions) relates to a measure where the total amount of dandruff on a defined area of human skin, preferably of human scalp, as observed (by a trained professional in the field) by human eye is reduced by more than 10, 20, 30, 40 50, 60, 70, 80 or 90% after one or repeated treatment of said defined area with a fatty acid ester or mixture or composition as defined herein.

The term “avoiding dandruff on human skin”, as used herein, relates to a preventive measure where the first occurrence or reoccurrence of dandruff on a defined area of human skin, preferably of human scalp, is avoided by applying a fatty acid ester or a mixture or a composition as defined herein once or repeatedly to said defined area of human skin. As a result, no dandruff or almost no dandruff is visible on said defined area of the human skin when inspected (by a trained professional in the field, such as e.g. a dermatologist) by human eye.

The terms “reducing the amount of Malassezia” or “treating an excess of Malassezia” as used herein is defined as a significant reduction of the total number of Malassezia cells on a defined area of skin surface of a mammal, preferably of a human, i.e. preferably a reduction of more than 10, 20, 30, 40, 50, 60, 70, 80 or 90% of the total number of Malassezia cells on a defined area of skin surface.

Moreover, the present invention relates to methods for therapeutically treating of an excess of Malassezia on the skin surface, preferably on the scalp, of mammals, preferably of humans, comprising the step of administering a fatty acid ester, as defined herein, a mixture, as defined herein, or a composition, as defined herein, to a mammal, preferably a human. Preferably, Malassezia is/are one or more species selected from the group consisting of M. globosa, M. restricta, M. pachydermatis and M. sympodialis. What is said herein with regard to the medical or cosmetic uses or the fatty acid esters, as defined herein, the mixtures, as defined herein, or the compositions, as defined herein, for a certain use in this regard, applies accordingly, as far as applicable.

Moreover, the present invention relates to methods for therapeutically treating or reducing dandruff on human skin, preferably on the human scalp, wherein the dandruff is caused by Malassezia, particularly preferably wherein Malassezia is/are one or more species selected from the group consisting of M. globosa, M. restricta, M. pachydermatis and M. sympodialis. What is said herein with regard to the medical or cosmetic uses or the fatty acid esters, as defined herein, the mixtures, as defined herein, or the compositions, as defined herein, for a certain use in this regard, applies accordingly, as far as applicable.

Further aspects and advantages of the present invention result from the subsequent description of preferred examples.

### EXAMPLES

### Example 1: Antifungal Activity of Known Compounds

As published in WO 2020/160741, the following MICs were observed for the compounds ethyl caprylate, 3-hydroxypropyl caprylate and glyceryl monocaprylate with regard to several Malassezia species.

| Test | Strain | | | | | | | |
|---|---|---|---|---|---|---|---|---|
| sub- | M. sympodialis | | | M. globosa | | M. restricta | | |
| stance | 7222 | 7979 | 42132 | 6171 | 7966 | PM1 | 7705 | 7877 |
| Ethyl | 1,000 | 1,000 | 1,000 | 1,000 | 1,000 | 1,000 | n, d, | 4,000 |
| capry- | | | | | | | | |
| late | | | | | | | | |
| 3-Hy- | 1,000 | 2,000 | 1,000 | 2,000 | 1,000 | 500 | 1,000 | 1,000 |
| droxy- | | | | | | | | |
| propyl | | | | | | | | |
| capry- | | | | | | | | |
| late | | | | | | | | |
| Glyceryl | 1,000 | 1,000 | 1,000 | 2,000 | 1,000 | 500- | 1,000 | 300 |
| mono- | | | | | | 1,000 | | |
| capry- | | | | | | | | |
| late | | | | | | | | |

### Example 2: Antifungal Activity According to the Invention

### 2.1 Microbial Test Strains

The fatty acid esters and their mixtures were tested for growth inhibition against Malassezia. The following reference strains were included:

| | |
|---|---|
| M. sympodialis | DSM 6171 |
| M. globosa | CBS 7966 |
| M. restricta | CBS 7877 |

CBS = Centraalbureau voor Schimmelcultures, Utrecht, NI;
DSM = DSMZ GmbH, Braunschweig, Germany

### 2.2 Preparation of Test Plates

Tests were performed in 6 well plates on solid media using agar dilution tests. Stock solutions of test substances were prepared in DMSO. Lower test concentrations were obtained by diluting stock solutions in DMSO.

### 2.3 Inoculation of Test Strains

Microbial test strains were prepared based on the procedure described by Mayser (Mayser P., Medium chain fatty acid ethyl esters-activation of antimicrobial effects by Malassezia enzymes. Mycoses 2015; 58:215-9). Briefly, microorganisms are inoculated on modified Leeming & Notman (MLN) agar. A well grown plate is harvested in a 0.8% NaCl solution. Dilutions of test substances are prepared according to the concentration range required for the test. 5 μl of the test sample and controls are complemented with 500 μl MLN agar and mixed by shaking. 200 μl of liquid MLN per well are added onto the solidified agar. Afterwards, 20 μl of inoculum solution is added, with except of the blanks, and samples are incubated for 7 days at 30° C. Typical concentrations to be tested are 5000 ppm, 2500 ppm, and 1:1 dilutions from 1000 ppm (500 ppm, 250 ppm, etc).

### 2.4 Results

Minimal inhibitory concentrations for test substances were determined for the different test strains, where each experiment is performed until at least two experiments showed both the minimal inhibitory concentration needed to inhibit growth (MIC) and maximal non-inhibiting concentrations. Appropriate controls are included to show the growth of organisms without antifungal substances and antifungal activity of reference substances.

As reference substances for antifungal activity, Climbazole (CAS 38083-17-9) and Piroctone olamine (CAS 68890-66-4) were used.

| | Strain | | |
|---|---|---|---|
| | M. sympodialis | M. globosa | M. restricta |
| Test substance | DSM 6171 | CBS 7966 | CBS 7877 |
| Xylityl sesquicaprylate | 2.500 | 1.000 | 2.500 |
| Polyglyceryl-3-caprylate | 2.500 | 1.000 | n.d. |
| Sorbitan caprylate | 5.000 | 1.000 | 2.500 |

The examples show that the esters, as defined herein, unexpectedly provided an antimycotic effect, which was in a similar range or even better than other esters with an already known antimycotic effect.

### Example 3: Formulation Examples

1=Protecting and scalp soothing hair conditioner, rinse off
2=Anti Dandruff shampoo
3=Natural beauty enhancer (solid concentrate)
4=Natural beauty protecting soft wet wipes

In formulations 1 to 4, the following perfume oils (fragrances) PO1 and PO2 were used. Each formulation was prepared with PO1 and with PO2 separately, wherein the same total amounts of PO1 or, respectively, PO2 were used.

Composition of perfume oil 1 (PO1, Amounts in ‰ b.w.)

| Ingredients | Amount |
|---|---|
| ALDEHYDE C14 SO-CALLED | 2 |
| ALLYL AMYL GLYCOLATE 10% DPG | 5 |
| ANISIC ALDEHYDE PURE | 5 |
| APPLE OLIFFAC TYPE | 10 |
| BENZYLACETATE | 50 |
| BERGAMOT IDENTOIL ® COLOURLESS | 15 |
| CANTHOXAL | 5 |
| CETALOX 10% IPM | 3 |
| CITRONELLOL 950 | 40 |
| DAMASCENONE TOTAL 1% DPG | 5 |
| DAMASCONE ALPHA 10% DPG | 5 |
| DAMASCONE DELTA 10% DPG | 2 |
| DIMETHYL BENZYL CARBINYL BUTYRATE | 2 |
| DIPROPYLENE GLYCOL | 178 |
| EBANOL | 2 |
| ETHYL DECADIENOATE TRANS CIS-2,4 10% IPM | 2 |
| FLOROSA | 5 |
| FRAMBINON ® 10% DPG (Raspberry Ketone) | 7 |
| GALAXOLIDE 50% IN IPM | 100 |
| GALBEX TYPE BASE | 1 |
| GERANYL ACETATE PURE | 2 |
| HEDIONE | 30 |
| HELIOTROPIN | 10 |
| HEXENYL ACETATE CIS-3 10% DPG | 1 |
| HEXENYL SALICYLATE CIS-3 | 5 |
| HEXYL CINNAMIC ALDEHYDE ALPHA | 70 |
| HEXYL SALICYLATE | 50 |
| HYDROXY CITRONELLAL | 10 |
| ISO E SUPER | 15 |
| ISORALDEINE 70 | 20 |
| LEAFOVERT ® | 1 |
| LILIAL | 60 |
| LINALOOL | 60 |
| LINALYL ACETATE | 20 |
| LYRAL | 7 |
| MANZANATE | 2 |
| PHENOXANOL | 7 |
| PHENYLETHYL ALCOHOL | 120 |
| SANDAL MYSORE CORE | 2 |
| SANDRANOL ® | 7 |
| STYRALYL ACETATE | 3 |
| TAGETES RCO 10% TEC | 2 |
| TERPINEOL PURE | 20 |
| TETRAHYDROGERANIOL 10% DPG | 5 |
| TONALIDE | 7 |
| VERTOCITRAL 10% DPG | 5 |
| VERTOFIX | 15 |
| Total | 1000 |

Composition of perfume oil 2 (PO2, Amounts in ‰ b.w.)

| Ingredients | Amount |
|---|---|
| AMBRETTOLIDE (MACRO) | 10 |
| AMBROXIDE 10% in IPM | 10 |
| BENZYL ACETATE | 20 |
| BENZYL SALICYLATE | 15 |
| BERGAMOT OIL. Bergapten-free | 60 |
| CALONE ® 1951 10% in DPG | 15 |
| COUMARIN | 5 |
| CYCLOGALBANATE ® 10% in DPG | 10 |
| ALPHA -DAMASCONE 1% in DPG | 20 |
| DIHYDROMYRCENOL | 10 |
| ETHYL LINALOOL | 75 |
| ETHYL LINALYLACETATE | 50 |
| ETHYL MALTOL 1% in DEP | 10 |
| ETHYLENE BRASSYLATE (MACRO) | 80 |
| FLOROSA | 40 |
| GERANYLACETATE | 10 |
| HEDIONE ® HC/30 | 35 |
| HEDIONE ® | 210 |
| HELIONAL ® | 15 |
| HELVETOLIDE ® (ALICYC) | 30 |
| HEXENYLSALICYLATE CIS-3 | 20 |
| ISO E SUPER ® | 40 |
| LEAFOVERT ® 10% in DEP | 10 |
| LILIAL ® | 80 |
| LYRAL ® | 20 |
| MANDARIN OIL | 10 |
| STYRALYL ACETATE | 5 |
| SYMROSE ® | 15 |
| VANILLIN 10% in DEP | 20 |
| DIPROPYLENE GLYCOL (DPG) | 50 |
| TOTAL | 1000 |

Cosmetic formulations 1 to 4 (amounts in % b.w.)

| | Amounts [wt.-%] | | | |
|---|---|---|---|---|
| Ingredients INCI name | 1 | 2 | 3 | 4 |
| Apricot Kernel Oil Prunus Armeniaca Kernel Oil | 0.5 | | | |
| Argan Oil Argania Spinosa Kernel Oil | 0.5 | | | |
| Beeswax White Cera Alba | | | 14.0 | |
| Benzyl Alcohol Benzyl Alcohol | | | 0.5 | |
| Carnauba Wax LC organic Copernicia Cerifera (Carnuba) | | | 6.0 | |
| Wax | | | | |
| Carvacrol | | | | 0.1 |
| Citric acid 10% solution Aqua. Citric acid | | | | 0.1 |
| Cocoa Butter Theobroma Cacao (Cocoa) Seed Butter | | | 3.0 | |
| Coconut oil Cocos Nucifera (Coconut) Oil | | | 1.0 | |
| Colour Colour | | 0.4 | | |
| Crinipan AD Climbazole | | 0.2 | | |
| Crinipan PMC Green Propanediol Caprylate | 1.0 | 0.1 | 0.5 | 0.3 |
| Cyclomethicone Cyclomethicone | | | | 0.2 |
| Dehyquart A CA Cetrimonium Chloride | 0.5 | | | |
| Dehyquart SP Quaternium-52 | 4.0 | | | |
| Dehyton PK 45 Cocamidopropyl Betaine | | 8.0 | | |
| Dimethicone Dimethicone | 0.1 | | | |
| Dissolvine GL-47S Tetrasodium Glutamate Diacetate | 0.2 | | | |
| Dracorin CE Glyceryl Stearate Citrate | 1.0 | | | |
| Dragosantol 100 Bisabolol | | | | 0.1 |
| EcoPres XC Xylityl Caprylate | 0.3 | 0.1 | | 0.6 |
| Emulgin B2 Ceteareth-20 | | | | 2.5 |
| PO1 or, respectively, PO2 Parfum | 0.5 | 1.0 | 0.5 | 0.1 |
| Frescolat ML cryst Menthyl Lactate | | 1.0 | | |
| Frescolat Plus Menthol. Menthyl Lactate | 0.3 | | | |
| Glycerin Glycerin | | | | 3.0 |
| Hydrolex E Ethylhexylglycerin | | | | 0.3 |
| Hydrolite 5 Pentylene Glycol | | | 2.0 | 3.0 |
| Hydrolite 6 1.2-Hexanediol | | 1.0 | | |
| Hydrolite 7 green 1.2-Heptanediol | | 1.0 | | |
| Hydrolite CG Caprylyl Glycol | | | 0.5 | 1.0 |
| Hydroviton ® PLUS 2290 Aqua. Pentylene glycol. Glycerin. | | | | 1.5 |
| Fructose.Urea. Citric acid. Sodium hydroxide. Maltose. Sodium | | | | |
| pca. Sodium chloride. Sodium lactate. Trehalose. Allantoin. | | | | |
| Sodium hyaluronate Glucose | | | | |
| ImerCare ® Pharma 00T Talc | | | 2.0 | |
| Isopropyl Myristate Isopropyl myristate | | | | 0.5 |
| Isopropyl Palmitate Isopropyl Palmitate | | | | 3.0 |
| Jaguar C-162 Hydroxypropyl guar hydroxypropyltrimonium | 0.3 | | | |
| chloride | | | | |
| Jojoba Oil Simmondsia Chinensis Seed Oil | | | | 0.5 |
| Lanette O Cetearyl Alcohol | | | | 2.5 |
| Lara Care A-200 Galactoarabinan | 0.5 | | | |
| Mango Butter Mangifera indica Seed Butter | | | Ad 100 | |
| Mineral oil Paraffinum Liquidum | | | | 4.0 |
| Nativacare 5600 Zea Mays (Corn Starch) | | 2.0 | | |
| Natriquest E30 Trisodiumn Ethylendiamine Disuccinate | | 0.3 | | |
| PCL Liquid 100 Cetearyl Ethylhexanoate | 0.3 | | | |
| PCL Solid Stearyl Heptanoate. Stearyl Caprylate | 3.0 | | | |
| Phytoconcentrole Hemp CBD Cannabis Sativa Seed Oil. | | | 0.5 | |
| Cannabidiol. Tocopherol | | | | |
| Plantacare 2000 UP decyl Glucoside | | 4.0 | | |
| Propylene Glycol Propylene Glycol | | 2.0 | | |
| Rice Bran Wax Oryza Sativa (Rice) Bran Wax | | | 6.0 | |
| Sodium Hydroxide 10% solution Aqua. Sodium Hydroxide | | 2.2 | | |
| Super Hartolan Lanolin Alcohol | | | | 0.2 |
| Sweet Almond Oil Prunus Amygdalus Dulcis (Sweet Almond) | | | 23.5 | |
| oil | | | | |
| SymBronze Caprylic/Capric Triglyceride. Isochrysis Galbana | | | 0.7 | |
| Extract | | | | |
| SymCalmin Butylene Glycol. Pentylene Glycol. | 0.3 | | | |
| Hydroxyphenyl Propamidobenzoic Acid | | | | |
| SymCare W2 PEG-40 Hydrogenated Castor Oil. Trideceth-9. | | | | 1.5 |
| Pentylene Glycol. 4-t-Butylcyclohexa-nol. Propylene Glycol. | | | | |
| Water. Hydroxyphenyl Propamidobenzoic Acid | | | | |
| SymClariol Decylene Glycol | | 0.5 | | |
| SymControl Care Aqua. Glycerin. Tetraselmis Suecica | | | | 1.0 |
| Extract | | | | |
| SymControl Scalp Aqua. Glycerin. Mannitol. Tetraselmis | 0.7 | | | |
| Suecica Extract | | | | |
| SymDecanox HA Caprylic/Capric Triglyceride. | | | 1.0 | |
| Hydroxymethoxyphenyl Decanone | | | | |
| SymDiol 68 1.2-Hexanediol. Caprylyl glycol | 0.5 | | | |
| SymGlucan Aqua. Glycerin. 1.2-Hexanediol. Caprylyl Glycol. | | | | 2.0 |
| Beta-Glucan | | | | |
| SymHair Force 1631 Pentylene Glycol. Isochrysis Galbana | | 1.0 | | |
| Extract | | | | |
| SymHair Restore Glycerin. Triticum vulgare protein. Aqua | 1.0 | | | |
| SymHair Shield Pentylene Glycol. Aqua. Glycerin. Triticum | | 1.0 | | |
| vulgare bran extract. 1.2-Hexanediol. Capryly glycol | | | | |
| SymLite G8 Glyceryl Caprylate | 0.2 | | | |
| SymOcide PS Phenoxyethanol. Decylene Glycol. 1.2- | 2.0 | | | |
| Hexanediol | | | | |
| SymReboot L19 Maltodextrin. Lactobacillus Ferment | | | 0.3 | |
| SymRenew ™ HPR Diisopropyl Adipate. Hydroxypinacolone | | | 1.0 | |
| Retinoate. Tocopherol | | | | |
| SymSave H Hydroxyacetophenone | | 0.5 | | |
| SymSoft Scalp Trideceth-9 (and) PEG-5 Ethylhexanoate | | 1.0 | | |
| (and) Water (and) Hydroxyphenyl Propamidobenzoic Acid | | | | |
| Tagat L2 PEG-20 Glyceryl Laurate | | | | 2.5 |
| Tego Cosmo P813 Polyglyceryl-3 Caprylate | | | 0.5 | |
| Texapon N70 Sodium Laureth Sulfate | | 35.0 | | |
| Water Aqua | Ad 100 | Ad 100 | | Ad 100 |
| Xanthan Gum Xanthan Gum | | | 0.2 | |
| Zinc omadine Zinc Pyrithione | | 0.3 | | |

1-15. (canceled)

16. A method for treating excess Malassezia on a surface of skin comprising applying to the surface of the skin an effective amount of one or more fatty acid esters, wherein the one or more fatty acid esters are esters of caprylic acid and a polyol, wherein the caprylic acid forms fatty acid residues of the one or more fatty acid esters and the polyol forms polyol residues of the one or more fatty acid esters, and the polyol residues carry at least 3 hydroxyl groups.

17. The method of claim 16, wherein the polyol residues carry 3 to 6 hydroxyl groups.

18. The method of claim 16, wherein the polyol residues carry 3 to 5 hydroxyl groups.

19. The method of claim 16, wherein the polyol residues carry 3 to 4 hydroxyl groups.

20. The method of claim 16, wherein the fatty polyol residues have at least 5 carbon atoms.

21. The method of claim 16, wherein the fatty polyol residues have from 5 to 10 carbon atoms.

22. The method of claim 16, wherein the one or more fatty acid esters are monoesters, diesters, or mixtures thereof.

23. The method of claim 16, wherein the one or more fatty acid esters are selected from xylityl caprylate, xylityl sesquicaprylate, polyglyceryl-3 caprylate, sorbitan caprylate, or mixtures thereof.

24. The method of claim 16, wherein the one or more fatty acid esters are selected from caprylyl caprylate, coco-caprylate/caprate, PEG-8 caprylate, dipropylene glycol caprylate, propylene glycol caprylate, pyridoxine dicaprylate, glyceryl caprylate/caprate, methyl glucose caprylate/caprate, polyglyceryl-3 caprate/caprylate/succinate, polyglyceryl-2 caprylate, polyglyceryl-3 caprylate, polyglyceryl-4 caprylate, polyglyceryl-6 caprylate, polyglyceryl-10 caprylate, polyglyceryl-4 caprylate/caprate, polyglyceryl-6 caprylate/caprate, polyglyceryl-10 caprylate/caprate, propanediol dicaprylate/caprate, xylityl caprate/caprylate, xylityl caprylate, or mixtures thereof.

25. The method of claim 16, wherein the one or more fatty acid esters are applied to the surface of the skin in a mixture.

26. The method of claim 16, wherein the Malassezia is selected from M. globosa, M. restricta, M. pachydermatis, and M. sympodialis, or mixtures thereof.

27. The method of claim 16, wherein the one or more fatty acid esters are applied to the surface of the skin in a composition and the composition is a personal care product.

28. The method of claim 27, wherein the composition further comprises:
(i) one or more 1,2-alkane diols;
(ii) one or more 2,3-alkane diols; and/or
(iii) one or more 1,3-alkane diols.

29. The method of claim 27, wherein the one or more fatty acid esters are in an amount of 0.001 to 25 wt. %, based on a total weight of the composition.

30. The method of claim 27, wherein the one or more fatty acid esters are in an amount of 0.01 to 10 wt. %, based on a total weight of the composition.

31. The method of claim 17, wherein the one or more fatty acid esters are in an amount of 0.05 to 3 wt. %, based on a total weight of the composition.

32. The method of claim 16, wherein the skin is skin of a human scalp.

33. The method of claim 32, wherein the method treats dandruff on a human scalp in need of treatment.

34. A composition comprising one or more fatty acid esters of caprylic acid and a polyol, in amount sufficient to treat an excess of Malassezia on a surface of skin;
wherein the caprylic acid forms fatty acid residues of the one or more fatty acid esters and the polyol forms polyol residues of the one or more fatty acid esters, and the polyol residues carry at least 3 hydroxyl groups; and
wherein the composition is a personal care product.

35. The composition of claim 34, wherein the one or more fatty acid esters are selected from xylityl caprylate, xylityl sesquicaprylate, polyglyceryl-3 caprylate, sorbitan caprylate, or mixtures thereof.
